(19) United States
(12) Patent Application Publication
(10) Pub. No.: US 2008/0306230 A1
(43) Pub. Date: Dec. 11, 2008

(54) Composition and Associated Method

(75) Inventors: Zhida Pan, Zhejiang (CN); Su Lu, Shanghai (CN); Liangliang Qiang, Shanghai (CN)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 11/759,333

(22) Filed: Jun. 7, 2007

(51) Int. Cl. B01J 31/22; C07D 207/00; C07D 327/00; C07F 3/08; C08F 4/72; C08F 4/80

(52) U.S. Cl. 526/171; 502/152; 544/181; 544/225; 546/4; 548/103; 548/402; 549/209; 549/3; 552/10; 556/136; 556/140; 556/20; 556/30; 556/7; 556/9; 585/520

(57) ABSTRACT

A composition includes a Group (VIII) transition metal, an anionic ligand bonded to the metal, a neutral electron donor ligand bonded to the metal, and an alkylidene group bonded to the metal. The alkylidene group includes a cycloaliphatic radical substituted with an ionic group. An associated method is also provided.

### BACKGROUND

1. Technical Field

The invention includes embodiments that relate to a metal complex composition. The invention includes embodiments that relate to a method of making the metal complex composition and method of catalyzing a metathesis reaction using the metal complex composition.

2. Discussion of Related Art

Metathesis reactions (for example, ring-closing metathesis or cross-metathesis reaction) may provide for synthesis of cyclic and heterocyclic molecules. Metathesis polymerization reactions (for example, ring opening metathesis polymerization or acyclic diene metathesis polymerization reaction) may provide for synthesis of functional polymers by controlled polymerization reaction.

A metal-complex (for example, a ruthenium alkylidine complex) may catalyze a metathesis reaction of an olefin. Metal-complexes employed as metathesis catalysts may not be soluble in polar solvents. Metal-complexes employed as metathesis catalysts may not provide the desired catalytic activity (reaction rate, product yield, and the like) or catalytic stability in a polar solvent, for example, in aqueous reaction conditions. For some applications (such as pharmaceutical or bioanalytical applications) aqueous-based reaction conditions may be needed and the metathesis reaction may have to be conducted in an aqueous environment.

It may be desirable to have metal complex compositions and methods for metathesis reactions that have characteristics that are different from those currently available.

### BRIEF DESCRIPTION

In one embodiment, a composition is provided. The composition includes a Group (VIII) transition metal, an anionic ligand bonded to the metal, a neutral electron donor ligand bonded to the metal, and an alkylidene group bonded to the metal. The alkylidene group includes a cycloaliphatic radical substituted with an ionic group.

In one embodiment, a composition is provided. The composition includes a Group (VIII) transition metal, an anionic ligand bonded to the metal, a neutral electron donor ligand bonded to the metal, and an alkylidene group bonded to the metal. The alkylidene group includes a cycloaliphatic radical substituted with an ionic group. The composition includes a polar solvent present in an amount sufficient that the composition is a slurry or a solution.

In one embodiment, an emulsion is provided. The emulsion includes a first phase having an olefin. The composition includes a second phase having a polar solvent and a composition having a Group (VIII) transition metal, an anionic ligand bonded to the metal, a neutral electron donor ligand bonded to the metal, and an alkylidene group bonded to the metal. The alkylidene group includes a cycloaliphatic radical substituted with an ionic group.

In one embodiment, an article is provided. The article includes a substrate having a surface and a composition adhered to the surface. The composition includes a second phase having a polar solvent and a composition having a Group (VIII) transition metal, an anionic ligand bonded to the metal, a neutral electron donor ligand bonded to the metal, and an alkylidene group bonded to the metal. The alkylidene group includes a cycloaliphatic radical substituted with an ionic group.

In one embodiment a composition is provided. The composition includes a reaction product of a composition having formula (XIV):

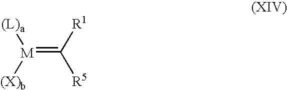

wherein “a” and “b” are independently integers from 1 to 3, with the proviso that “a+b” is less than or equal to 5, M is ruthenium or osmium, X is independently at each occurrence an anionic ligand, L is independently at each occurrence a neutral electron donor ligand, R¹ is hydrogen, an aliphatic radical, a cycloaliphatic radical, or an aromatic radical, and R⁵ is hydrogen, an aliphatic radical, or an aromatic radical; and a cycloolefin having an ionic group, or a polynorbornene having an ionic group and a pendent alkene group, wherein the ionic group includes one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium.

In one embodiment a method is provided. The method includes contacting a composition having formula (XIV)

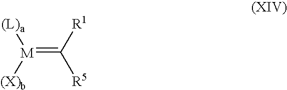

wherein “a” and “b” are independently integers from 1 to 3, with the proviso that “a+b” is less than or equal to 5, M is ruthenium or osmium, X is independently at each occurrence an anionic ligand, L is independently at each occurrence a neutral electron donor ligand, R¹ is hydrogen, an aliphatic radical, a cycloaliphatic radical, or an aromatic radical, and R⁵ is hydrogen, an aliphatic radical, or an aromatic radical; with a cycloolefin having an ionic group, or a polynorbornene having an ionic group and a pendent alkene group, wherein the ionic group includes one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium.

### BRIEF DESCRIPTION OF DRAWING FIGURES

FIG. 1 is a reaction scheme for synthesis of norbornene-sulfonate.

FIG. 2 is a proton NMR spectrum of norbornene sulfonate.

FIG. 3 is a reaction scheme for synthesis of norbornene-gluconate.

FIG. 4 is a proton NMR spectrum of norbornene gluconate.

FIG. 5 is a reaction scheme for synthesis of a water-soluble metal complex.

FIG. 6 is a proton NMR spectrum of a water-soluble metal complex.

FIG. 7 is a proton NMR spectrum showing the alkylidine signal of a water-soluble metal complex.

FIG. 8 is a proton NMR spectrum of a polymer formed using the water-soluble metal complex.

FIG. 9 is a plot of polymer yield as a function of polymer precursor to catalyst ratio.

FIG. 10 is a schematic illustration of preparation of a surface-supported metal complex.

FIG. 11 shows the fluorescence images of slides prepared in Example 6.

FIG. 12 shows the fluorescence images of slides prepared in Example 6.

FIG. 13 shows the fluorescence images of slides prepared in Example 6.

FIG. 14 is an absorbance spectra of slides prepared in Example 6.

### DETAILED DESCRIPTION

The invention includes embodiments that relate to a metal complex composition. The invention includes embodiments that relate to a method of making the metal complex composition and method of catalyzing a metathesis reaction using the metal complex composition.

In the following specification and the clauses which follow, reference will be made to a number of terms have the following meanings. The singular forms “a”, “an” and “the” include plural referents unless the context clearly dictates otherwise. Approximating language, as used herein throughout the specification and clauses, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Similarly, “free” may be used in combination with a term, and may include an insubstantial number, or trace amounts, while still being considered free of the modified term.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity can not occur—this distinction is captured by the terms “may” and “may be”.

The invention includes a composition in one embodiment. The composition includes a Group (VIII) transition metal, an anionic ligand bonded to the metal, a neutral electron donor ligand bonded to the metal, and an alkylidene group bonded to the metal. The alkylidene group includes a cycloaliphatic radical substituted with an ionic group. In one embodiment, the composition as described hereinabove may form a metal complex.

As used herein, the term “transition metal” refers to an element whose atom has an incomplete d sub-shell, or which can give rise to cations with an incomplete d sub-shell. As used herein, the term “Group (VIII) transition metal” refers to transition metals in the Group (VIII) of the periodic table. In one embodiment, a Group (VIII) transition metal may include ruthenium (Ru) or Osmium (Os). In one embodiment, ruthenium or osmium may form a metal center of the metal complex.

In one embodiment, the Group (VIII) transition metal in the metal complex may be in the +2 oxidation state, may have an electron count of 16, and may be penta-coordinated. For example, a metal complex may include a ruthenium or osmium metal center that is in the +2 oxidation state, has an electron count of 16, and is penta-coordinated. In an alternate embodiment, the Group (VIII) transition metal in the metal complex may be in the +2 oxidation state, may have an electron count of 18, and may be hexa-coordinated. For example, a metal complex may include a ruthenium or osmium metal center that is in the +2 oxidation state, has an electron count of 18, and is hexa-coordinated.

Independent of the type of Group (VIII) transition metal, a composition may include one or more neutral electron-donating ligand, one or more anionic ligand, and an alkylidene radical. A neutral electron-donating ligand, an anionic ligand or an alkylidene radical may be bonded to the metal center by coordination bond formation. As used herein, the term “neutral electron-donating ligand” refers to ligands that have a neutral charge when removed from the metal center. As used herein, the term “anionic ligand” refers to ligands that are negatively charged when removed from the metal center. As used herein the term “alkylidene radical” refers to a substituted or unsubstituted divalent organic radical formed from an alkane by removal of two hydrogen atoms from the same carbon atom, the free valencies of which are part of a double bond. In one embodiment, a carbon atom in the alkylidene radical may form a double bond with the metal center in the metal complex. In one embodiment, the metal bonded carbon atom of the alkylidene radical may be substituted with one or more cycloaliphatic radical. Cycloaliphatic radical may be defined as the following:

A cycloaliphatic radical is a radical having a valence of at least one, and having an array of atoms, which is cyclic but which is not aromatic. A cycloaliphatic radical may include one or more non-cyclic components. For example, a cyclohexylmethyl group (C₆H₁₁CH₂—) is a cycloaliphatic radical, which includes a cyclohexyl ring (the array of atoms, which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. The term “a C₃-C₃₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical. In one embodiment, a cycloaliphatic radical essentially includes a cyclopentyl radical, which is a cyclic array of 5 atoms. The cyclopentyl radical may include heteroatoms such as nitrogen, sulfur, selenium, silicon and oxygen, or may be composed exclusively of carbon and hydrogen.

A cycloaliphatic radical is substituted with one or more ionic group. An ionic group may include a positively charged or a negatively charged functional group. In one embodiment, a cycloaliphatic radical may be substituted with one or more anionic group. Anionic groups may include phosphite, phosphate, sulfite, sulfate, sulfonate, nitrite, nitrate, azide, or carboxylate. In one embodiment, a cycloaliphatic radical may be substituted with one or more cationic group. Cationic groups may include ammonium, quaternary ammonium, phosphonium, quaternary phosphonium, imidazolium, pyridinium, or sulfonium. In one embodiment, a cycloaliphatic radical may be substituted directly with an ionic group. For example, a cycloaliphatic radical may be directly substituted with a COO⁻ anion.

In one embodiment, an ionic group may include an aliphatic radical, a cycloaliphatic radical a cycloaliphatic radical and a positively charged or a negatively charged functional group. For example, an ionic group may include a —CH₂COO— anion or —C₆H₄COO— anion. Aliphatic radical and an aromatic radical may be defined as the following:

Aliphatic radical is an organic radical having at least one carbon atom, a valence of at least one and may be a linear or branched array of atoms. Aliphatic radicals may include heteroatoms such as nitrogen, sulfur, silicon, selenium and oxygen or may be composed exclusively of carbon and hydrogen. Aliphatic radical may include a wide range of functional groups such as alkyl groups, alkenyl groups, alkynyl groups, halo alkyl groups, conjugated dienyl groups, alcohol groups, ether groups, aldehyde groups, ketone groups, carboxylic acid groups, acyl groups (for example, carboxylic acid derivatives such as esters and amides), amine groups, nitro groups and the like. For example, the 4-methylpent-1-yl radical is a C₆ aliphatic radical comprising a methyl group, the methyl group being a functional group, which is an alkyl group. Similarly, the 4-nitrobut-1-yl group is a C₄ aliphatic radical comprising a nitro group, the nitro group being a functional group. An aliphatic radical may be a haloalkyl group that includes one or more halogen atoms, which may be the same or different. Halogen atoms include, for example; fluorine, chlorine, bromine, and iodine. Aliphatic radicals having one or more halogen atoms include the alkyl halides: trifluoromethyl, bromodifluoromethyl, chlorodifluoromethyl, hexafluoroisopropylidene, chloromethyl, difluorovinylidene, trichloromethyl, bromodichloromethyl, bromoethyl, 2-bromotrimethylene (e.g., —CH₂CHBrCH₂—), and the like. Further examples of aliphatic radicals include allyl, aminocarbonyl (—CONH₂), carbonyl, dicyanoisopropylidene —CH₂C(CN)₂CH₂—), methyl (—CH₃), methylene (—CH₂—), ethyl, ethylene, formyl (—CHO), hexyl, hexamethylene, hydroxymethyl (—CH₂OH), mercaptomethyl (—CH₂SH), methylthio (—SCH₃), methylthiomethyl (—CH₂SCH₃), methoxy, methoxycarbonyl (CH₃OCO—), nitromethyl (—CH₂NO₂), thiocarbonyl, trimethylsilyl ((CH₃)₃Si—), t-butyldimethylsilyl, trimethoxysilylpropyl ((CH₃O)₃SiCH₂CH₂CH₂—), vinyl, vinylidene, and the like. By way of further example, a “C₁-C₃₀ aliphatic radical” contains at least one but no more than 30 carbon atoms. A methyl group (CH₃—) is an example of a C₁ aliphatic radical. A decyl group (CH₃(CH₂)₉—) is an example of a C₁₀ aliphatic radical.

An aromatic radical is an array of atoms having a valence of at least one and having at least one aromatic group. This may include heteroatoms such as nitrogen, sulfur, selenium, silicon and oxygen, or may be composed exclusively of carbon and hydrogen. Suitable aromatic radicals may include phenyl, pyridyl, furanyl, thienyl, naphthyl, phenylene, and biphenyl radicals. The aromatic group may be a cyclic structure having 4 n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), anthracenyl groups (n=3) and the like. The aromatic radical also may include non-aromatic components. For example, a benzyl group may be an aromatic radical, which includes a phenyl ring (the aromatic group) and a methylene group (the non-aromatic component). Similarly a tetrahydronaphthyl radical is an aromatic radical comprising an aromatic group (C₆H₃) fused to a non-aromatic component —(CH₂)₄—. An aromatic radical may include one or more functional groups, such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group, which is an alkyl group. Similarly, the 2-nitrophenyl group is a C6 aromatic radical comprising a nitro group, the nitro group being a functional group. Aromatic radicals include halogenated aromatic radicals such as trifluoromethylphenyl, hexafluoroisopropylidenebis(4-phen-1-yloxy) (—OPhC(CF₃)₂PhO—), chloromethylphenyl, 3-trifluorovinyl-2-thienyl, 3-trichloromethylphen-1-yl (3-CCl₃Ph—), 4-(3-bromoprop-1-yl)phen-1-yl (BrCH₂CH₂CH₂Ph—), and the like. Further examples of aromatic radicals include 4-allyloxyphen-1-oxy, 4-aminophen-1-yl (H₂NPh—), 3-aminocarbonylphhen-1-yl (NH₂COPh—), 4-benzoylphen-1-yl, dicyanoisopropylidenebis(4-phen-1-yloxy) (—OPhC(CN)₂PhO—), 3-methylphen-1-yl, methylenebis(phen-4-yloxy) (—OPhCH₂PhO—), 2-ethylphen-1-yl, phenylethenyl, 3-formyl-2-thienyl, 2-hexyl-5-furanyl; hexamethylene-1,6-bis(phen-4-yloxy) (—OPh(CH₂)₆PhO—), 4-hydroxymethylphen-1-yl (4-HOCH₂Ph—), 4-mercaptomethylphen-1-yl (4-HSCH₂Ph—), 4-methylthiophen-1-yl (4-CH₃SPh—), 3-methoxyphen-1-yl, 2-methoxycarbonylphen-1-yloxy (e.g., methyl salicyl), 2-nitromethylphen-1-yl (—PhCH₂NO₂), 3-trimethylsilylphen-1-yl, 4-t-butyldimethylsilylphenl-1-yl, 4-vinylphen-1-yl, vinylidenebis(phenyl), and the like. The term “a C₃-C₃₀ aromatic radical” includes aromatic radicals containing at least three but no more than 30 carbon atoms. The aromatic radical 1-imidazolyl (C₃H₂N₂—) represents a C₃ aromatic radical. The benzyl radical (C₇H₇—) represents a C₇ aromatic radical.

In one embodiment, an ionic group may include an aliphatic radical and a negatively charged or a positively charged group. In one embodiment, an ionic group may include a negatively charged or a positively charge derivative of an amino acid or a peptide. For example, an ionic group may include an aspartate group or a glutamate group. In one embodiment, an ionic group may include a negatively charged or a positively charged derivative of a carbohydrate. For example, an ionic group may include a gluconate group, which is a carboxylate derivative of glucose. Peptide and carbohydrate may be defined as the following:

As used herein, the term “peptide” refers to a linear sequence of amino acids connected to the other by peptide bonds between the alpha amino and carboxyl groups of adjacent amino acids. The amino acids may be the standard amino acids or some other non standard amino acids. Some of the standard nonpolar (hydrophobic) amino acids include alanine (Ala), leucine (Leu), isoleucine (Ile), valine (Val), proline (Pro), phenylalanine (Phe), tryptophan (Trp) and methionine (Met). The polar neutral amino acids include glycine (Gly), serine (Ser), threonine (Thr), cysteine (Cys), tyrosine (Tyr), asparagine (Asn) and glutamine (Gln). The positively charged (basic) amino acids include arginine (Arg), lysine (Lys) and histidine (His). The negatively charged (acidic) amino acids include aspartic acid (Asp) and glutamic acid (Glu). The non standard amino acids may be formed in body, for example by posttranslational modification, some examples of such amino acids being selenocysteine and pyrolysine. The peptides may be of a variety of lengths, either in their neutral (uncharged) form or in forms such as their salts. The peptides may be either free of modifications such as glycosylations, side chain oxidation or phosphorylation or comprising such modifications. Substitutes for an amino acid within the sequence may also be selected from other members of the class to which the amino acid belongs. A suitable peptide may also include peptides modified by additional substituents attached to the amino side chains, such as glycosyl units, lipids or inorganic ions such as phosphates as well as chemical modifications of the chains. Thus, the term “peptide” or its equivalent may be intended to include the appropriate amino acid sequence referenced, subject to the foregoing modifications, which do not destroy its functionality.

As used herein, the term “carbohydrate” refers to a polyhydroxy aldehyde or ketone, or a compound that can be derived from them by any of several means including (1) reduction to give sugar alcohols; (2) oxidation to give sugar acids; (3) substitution of one or more of the hydroxyl groups by various chemical groups, for example, hydrogen may be substituted to give deoxysugars, and amino group (NH2 or acetyl-NH) may be substituted to give amino sugars; (4) derivatization of the hydroxyl groups by various moieties, for example, phosphoric acid to give phosphor sugars, or sulphuric acid to give sulfo sugars, or reaction of the hydroxyl groups with alcohols to give monosaccharides, disaccharides, oligosaccharides, and polysaccharides. Carbohydrate group may include monosaccharides, disaccharides, or oligosaccharides. Suitable monosachharides may include, but are not limited to, glucose, fructose, mannose and galactose. A disachharide, as further defined herein, is a compound, which upon hydrolysis yields two molecules of a monosachharide. Suitable disachharides may include, but are not limited to, lactose, maltose, isomaltose, trehalose, maltulose, and sucrose. Suitable oligosachharides may include, but are not limited to, raffinose and acarbose. Also included are the sachharides modified by additional substituents, for example, methyl glycosides, N-acetyl-glucosamine, N-acetyl-galactosamine and their de-acetylated forms.

In one embodiment, a cycloaliphatic radical may be substituted with one or more neutral functional group. A neutral functional group may include one or more of a halogen atom, an aliphatic radical, a cycloaliphatic radical, an aromatic radical, an alkoxy group, a hydroxy group, an ether group, an aldehyde group, a ketone group, a silanyl group, a phosphanyl group, an amine group, or a nitro group. Halogen atoms include, for example, fluorine, chlorine, bromine, and iodine.

In one embodiment, a cycloaliphatic radical may include one or more cyclopentyl radical substituted with one or more ionic groups. In one embodiment, a cycloaliphatic radical may include one or more cyclopentyl radical substituted with one or more ionic groups and one or more neutral functional groups.

In one embodiment, a metal complex may have a formula (I):

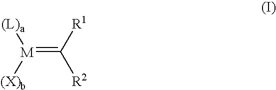

wherein “a” and “b” may be independently integers from 1 to 3, with the proviso that “a+b” may be less than or equal to 5;
M may be ruthenium or osmium;
X may be independently at each occurrence an anionic ligand;
L may be independently at each occurrence a neutral electron donor ligand;
R¹ may be hydrogen, an aliphatic radical, a cycloaliphatic radical, or an aromatic radical; and
R² may include one or more cycloaliphatic radical, and the cycloaliphatic radical may be substituted with an ionic group, wherein the ionic group may include one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium.

In one embodiment, R² may include one or more cyclopentyl radical substituted with one or more ionic groups. In one embodiment, R² may include one or more cyclopentyl radical substituted with one or more ionic groups and one or more neutral functional groups. In one embodiment, R² may include structural units having a formula (II) or (III)

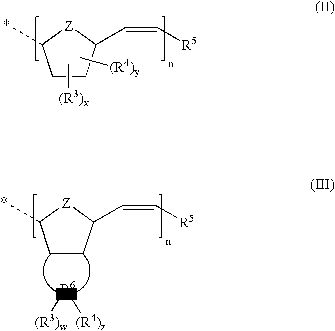

wherein * indicates the position where R² may be bonded to the carbon atom of the alkylidene group;
“w” may be 0, 1, 2, or 3, “x” may be 0 or 1, “y” may be 1 or 2, “z” may be 1, 2, 3, or 4, “n” may be an integer of from 1 to 100;
R³ may be independently at each occurrence hydrogen, a halogen atom, an aliphatic radical, a cycloaliphatic radical, an aromatic radical, an alkoxy group, a hydroxy group, an ether group, an aldehyde group, a ketone group, a silanyl group, a phosphanyl group, an amine group, a nitro group, or a divalent bond linking two carbon atoms;
R⁴ may be independently at each occurrence an ionic group, wherein the ionic group includes one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium;
R⁵ may be hydrogen, an aliphatic radical, a cycloaliphatic radical, or an aromatic radical;
R⁶ may be an aliphatic or an aromatic cyclic ring; and
Z may be C(R⁷)₂, C═C(R⁷)₂, Si(R⁷)2, O, S, N—R⁷, P—R⁷, B—R⁷, or As—R⁷ wherein R⁷ may be independently at each occurrence hydrogen, an aliphatic radical, a cycloaliphatic radical, or an aromatic radical. Aliphatic radical, cycloaliphatic radical, and an aromatic radical are as defined hereinabove.

In one embodiment, R² may include structural units having a formula (IV) or (V)

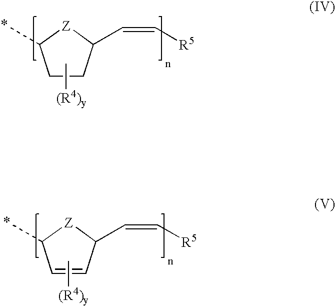

wherein “y”, “n”, R⁴, R⁵, and Z are as defined hereinabove.

In one embodiment, R² may include structural units having a formula (VI) or (VII)

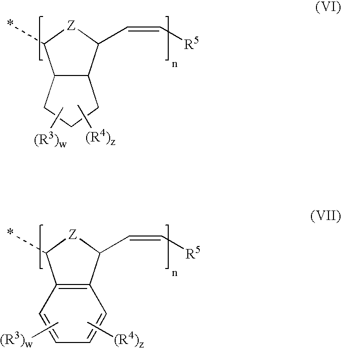

wherein “w”, “y”, “n”, R³, R⁴, R⁵, and Z are as defined hereinabove.

In one embodiment, R² may include structural units having a formula (VIII)

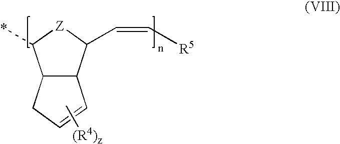

wherein “z”, “n”, R⁴, R⁵, and Z are as defined hereinabove.

In one embodiment, R² may include structural units having a formula (IX) or (X)

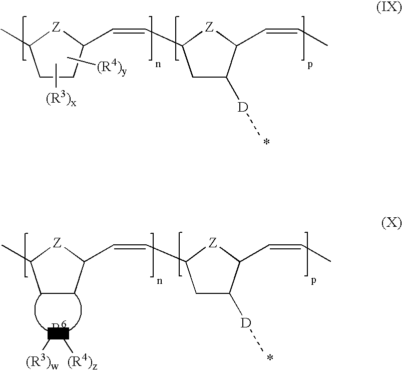

wherein * indicates the position where R² may be bonded to the carbon atom of the alkylidene group; “w”, “x”, “y”, “n”, R³, R⁴, R⁵, R⁶, and Z are as defined hereinabove; “p” is an integer in a range of from about 1 to about 100; and D is a divalent aromatic radical.

In one embodiment, D may include a divalent aromatic radical. In one embodiment, D may include a divalent aromatic radical and one or more divalent aliphatic radical —R⁸—, —R⁸O—, —R⁸CO₂—, —R⁸OCO—, R⁸CO₂R⁸—, —R⁸CO₂NR⁹—, —R⁸NR⁹CO—, —R⁸CONR⁹R⁸—, or —R⁸NR⁹COR⁸—, wherein R⁸ is a C₁-C₂₀ aliphatic radical and R⁹ is hydrogen or C₁-C₁₀ aliphatic radical. In one embodiment, D may include a divalent benzyl radical and one or more of the aforementioned divalent aliphatic radical.

In one embodiment, Z essentially includes a C(R⁷)₂ group. In one embodiment, Z essentially includes a CH₂ group. In one embodiment, Z essentially includes an O atom. In one embodiment, R⁵ essentially includes an aromatic radical. In one embodiment, R⁵ essentially includes a substituted or an unsubstituted benzyl radical. In one embodiment, R⁴ essentially includes an aliphatic radical substituted with one or more ionic group. In one embodiment, R⁴ essentially includes an aliphatic radical having an ether linkage and substituted with one or more ionic group.

The number of repeat units or the number of cycloaliphatic radicals in R² may be defined as chain length or “n”. In one embodiment, “n” may be in a range that is greater than about 1. In one embodiment, “n” may be in a range of from about 1 to about 5, from about 5 to about 10, from about 10 to about 20, from about 20 to about 40, from about 40 to about 60, from about 60 to about 80, or from about 80 to about 100. In one embodiment, “n” is essentially in a range of from about 10 to about 20. In one embodiment, “n+p” may be in a range that is greater than about 2. In one embodiment, “n+p” may be in a range of from about 2 to about 5, from about 5 to about 10, from about 10 to about 20, from about 20 to about 40, from about 40 to about 60, from about 60 to about 80, or from about 80 to about 100. In one embodiment, “n+p” is essentially in a range of from about 10 to about 20. Here and throughout the specification and claims, range limitations may be combined and/or interchanged. Such ranges as identified include all the sub-ranges contained therein unless context or language indicates otherwise.

A metal complex may include one or more anionic ligand X as described hereinabove in formula (I). An anionic ligand X may be a unidentate ligand or bidentate ligand. In one embodiment, X in formula (I) may be independently at each occurrence a halide, a carboxylate, a sulfonate, a sulfonyl, a sulfinyl, a diketonate, an alkoxide, an aryloxide, a cyclopentadienyl, a cyanide, a cyanate, a thiocyanate, an isocyanate, or an isothiocyanate. In one embodiment, X in formula (I) may be independently at each occurrence chloride, fluoride, bromide, iodide, CF₃CO₂, CH₃CO₂, CFH₂CO₂, (CH₃)₃CO, (CF₃)₂(CH₃)CO, (CF₃)(CH₃)₂CO, PhO, MeO, EtO, tosylate, mesylate, or trifluoromethanesulfonate.

The number of anionic ligands X bonded to the transition metal may depend on one or more of the coordination state of the transition metal (for example, penta-coordinated or hexa-coordinated), the number of neutral electron donating ligands bonded to the transition metal, or dentency of the anionic ligand. In one embodiment, X in formula (I) may include a unidentate anionic ligand and “b” may be 2. In one embodiment, X in formula (I) may include a bidentate anionic ligand and “b” may be 1. In one embodiment, X in formula (I) may be independently at each occurrence a chloride and “b” may be 2.

A composition may include one or more neutral electron donor ligand L as described hereinabove in formula (I). In one embodiment, L may be independently at each occurrence a monodentate, a bidentate, a tridentate, or a tetradentate neutral electron donor ligand.

In one embodiment, at least one L may be phosphine, phosphite, phosphinite, phosphonite, arsine, stibine, ether, amine, amide, imine, sulfoxide, carboxyl, nitrosyl, or thioethene. In one embodiment, at least one L may be a phosphine having formula P(R¹⁰R¹¹R¹²), where R¹⁰, R¹¹, and R¹² are each independently an aliphatic radical, a cycloaliphatic radical, or an aromatic radical. In one embodiment, at least L may include P(cyclohexyl)₃, P(cyclopentyl)₃, P(isopropyl)₃, or P(phenyl)₃.

In one embodiment, at least one L may be a heterocyclic ligand. A heterocyclic ligand refers to an array of atoms forming a ring structure and including one or more heteroatoms as part of the ring. A heteroatom is an atom other than carbon and hydrogen, and may include the group 15 or group 16 atom of the periodic table. In one embodiment, a heteroatom may include N, O, P, S, As or Se atoms. A heterocyclic ligand may be aromatic (heteroarene ligand) or non-aromatic, wherein a non-aromatic heterocyclic ligand may be saturated or unsaturated. A heterocyclic ligand may be further fused to one or more cyclic ligand, which may be a heterocycle or a cyclic hydrocarbon, for example in indole.

In one embodiment, at least one L may be a heteroarene ligand. A heteroarene ligand refers to an unsaturated heterocyclic ligand in which the double bonds form an aromatic system. In one embodiment, at least one L is furan, thiophene, pyrrole, pyridine, bipyridine, picolylimine, gamma-pyran, gamma-thiopyran, phenanthroline, pyrimidine, bipyrimidine, pyrazine, indole, coumarone, thionaphthene, carbazole, dibenzofuran, dibenzothiophene, pyrazole, imidazole, benzimidazole, oxazole, thiazole, dithiazole, isoxazole, isothiazole, quinoline, bisquinoline, isoquinoline, bisisoquinoline, acridine, chromene, phenazine, phenoxazine, phenothiazine, triazine, thianthrene, purine, bisimidazole, or bisoxazole. In one embodiment, at least one L may be a monodentate heteroarene ligand, which may be unsubstituted or substituted, for example, pyridine. In one embodiment at least one L may be a bidentate heteroarene ligand, which may be substituted or unsubstituted, for example, bipyridine, phenanthroline, bithiazole, bipyrimidine, or picolylimine.

In one embodiment, at least one L may be a N-heterocyclic carbene ligand (NHC). A N-heterocyclic carbene ligand is a heterocyclic ligand including at least one N atom in the ring and a carbon atom having a free electron pair. Examples of NHC ligands may include ligands of formula (XI), (XII), or (XII)

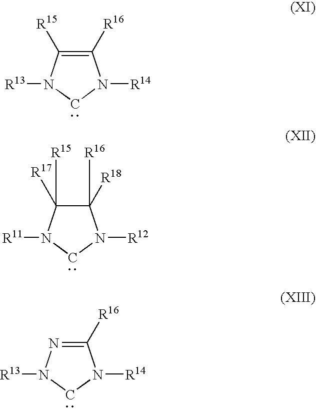

wherein R¹³, R¹⁴, R¹⁵, R¹⁶, R¹⁷, or R¹⁸ may be independently at each occurrence hydrogen, an aliphatic radical, a cycloaliphatic radical, or an aromatic radical. In one embodiment, R¹⁵, R¹⁶, R¹⁷, and R¹⁸ may be independently at each occurrence hydrogen. In one embodiment, R¹³ and R¹⁴ may be independently at each occurrence a substituted or an unsubstituted aromatic radical.

In one embodiment, a N-heterocyclic carbene ligand may include 1,3-dimesitylimidazolidin-2-ylidene, 1,3-di(1-adamantyl)imidazolidin-2-ylidene, 1-cyclohexyl-3-mesitylimidazolidin-2-ylidene, 1,3-dimesityloctahydrobenzimidazol-2-ylidene, 1,3-diisopropyl-4-imidazolin-2-ylidene, 1,3-di(1-phenylethyl)-4-imidazolin-2-ylidene, 1,3-dimesityl-2,3-dihydrobenzimidazol-2-ylidene, 1,3,4-triphenyl-2,3,4,5-tetrahydro-1H-1,2,4-triazol-5-ylidene, 1,3-dicyclohexylhexahydropyrimidin-2-ylidene, N,N,N′,N′-tetraisopropylformamidinylidene, 1,3,4-triphenyl-4,5-dihydro-1H-1,2,4-triazol-5-ylidene, or 3-(2,6-diisopropylphenyl)-2,3-dihydrothiazol-2-ylidene.

The number of neutral electron donor ligands L bonded to the transition metal may depend on one or more of the coordination state of the transition metal (for example, penta-coordinated or hexa-coordinated), the number of anionic ligands bonded to the transition metal, or dentency of the neutral electron donor ligand. In one embodiment, “a” in formula (I) may be 1. In one embodiment, “a” in formula (I) may be 2. In one embodiment, “a” in formula (I) may be 3. In one embodiment, R¹, R², X and L may be bound to one another in an arbitrary combination to form a multidentate chelate ligand.

In one embodiment, a metal complex may be prepared by contacting a composition having a formula (XIV)

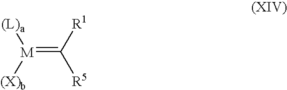

with a cycloolefin having an ionic group. In one embodiment, a metal complex may be prepared by contacting a composition having a formula (XIV) with a polynorbornene having an ionic group and a pendent alkene group, wherein “a”, “b”, M, X, L, R¹, and R⁵ are as defined hereinabove.

A cycloolefin refers to a non-aromatic cyclic ring having at least one carbon-carbon double bond in the cyclic ring. In one embodiment, a cyclic olefin may include a strained cyclic structure (for example, norbornene). In one embodiment, a cycloolefin may include one or more heteroatoms (for example, oxanorbornene), where heteroatoms are as defined hereinabove. In one embodiment, a cycloolefin may include at least two carbon-carbon double bonds (for example, norbornadiene). In one embodiment, a cycloolefin may include two or more cyclic rings that may be fused with each other (for example, dicyclopentadiene). A cycloolefin may be substituted with at least one ionic group, and the ionic group includes one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium.

In one embodiment, a suitable ionic group-substituted cycloolefin may include at least one norbornene group having a formula (XV) or (XVI)

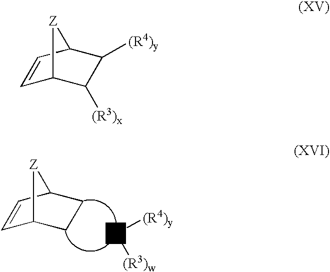

wherein “w”, “x”, “y”, R³, R⁴, R⁵, R⁶ and Z are as defined hereinabove.

The term “polynorbornene”, as used herein, may include a polymer having one or more structural units derived from a cycloolefin having a formula (XV) or (XVI). In one embodiment, a suitable ionic group-substituted polynorbornene may include structural units having a formula (XVII) or (XVIII)

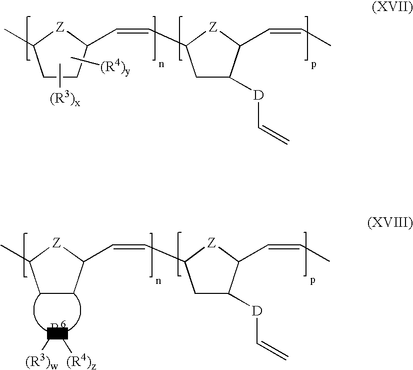

wherein “w”, “x”, “y”, “n”, “p”, R³, R⁴, R⁵, R⁶, D, and Z are as defined hereinabove.

In one embodiment, at least one L in formula (XIV) may include a phosphine ligand. In one embodiment, at least one L in formula (XIV) may include P(cyclohexyl)₃, P(cyclopentyl)₃, P(isopropyl)₃, or P(phenyl)₃. In one embodiment, at least one L in formula (XIV) may include P(cyclohexyl)₃. In one embodiment, at least one L in formula (XIV) may include a monodentate pyridine ligand, which is unsubstituted or substituted. In one embodiment, at least one L in formula (XIV) may include a bromine-substituted monodentate pyridine ligand. In one embodiment, at least one L in formula (XIV) may include a N-heterocyclic carbene ligand (NHC). In one embodiment, at least one L in formula (XIV) may include an NHC ligands having formula (XI), (XII), or (XIII).

In one embodiment, R⁵ in formula (XIV) may include an aromatic radical. In one embodiment, R⁵ in formula (XIV) may include a substituted or an unsubstituted benzyl radical. In one embodiment, at least one X in formula (XIV) may include a halide. In one embodiment, at least one X in formula (XIV) may include a chloride.

In one embodiment, a composition having a formula (XIV) may include Bis(tricyclohexylphosphine) benzylidine ruthenium(IV) chloride (CAS No. 172222-30-9), 1,3-Bis(2,4,6-trimethylphenyl)-2-imidazolidinylidene)dichloro(phenylmethylene)(tricyclohexylphosphine)ruthenium (CAS No. 246047-72-3), 1,3-Bis(2,4,6-trimethylphenyl)-2-imidazolidinylidene)dichloro(phenylmethylene)(di-3-bromopyridine)ruthenium, or 1,3-Bis-(2,4,6-trimethylphenyl)-2-imidazolidinylidene)dichloro(o-isopropoxyphenylmethylene)ruthenium (CAS No. 301224-40-8).

In one embodiment, a reaction product of a composition having formula (XIV) and an ionic-group substituted cycloolefin or an ionic group-substituted polynorbornene is provided. In one embodiment, the conversion of the cycloolefin may be complete, that is, the reaction product may be free of any unreacted cycloolefin. In one embodiment, the conversion of the cycloolefin may be incomplete, that is, the reaction product may include unreacted cycloolefin. In one embodiment, the conversion of the cycloolefin may be in a range that is greater than about 50 weight percent. In one embodiment, the conversion of the cycloolefin may be in a range of from about 50 weight percent to about 60 weight percent, from about 60 weight percent to about 70 weight percent, from about 70 weight percent to about 80 weight percent, from about 80 weight percent to about 90 weight percent, or from about 90 weight percent to about 100 weight percent. In one embodiment, a reaction product may include a metal complex having formula (I), unreacted cycloolefin, and unreacted composition having formula (XIV).

In one embodiment, the conversion of the polynorbornene may be complete, that is, the reaction product may be free of any unreacted polynorbornene. In one embodiment, the conversion of the polynorbornene may be incomplete, that is, the reaction product may include unreacted polynorbornene. In one embodiment, the conversion of the polynorbornene may be in a range that is greater than about 50 weight percent. In one embodiment, the conversion of the polynorbornene may be in a range of from about 50 weight percent to about 60 weight percent, from about 60 weight percent to about 70 weight percent, from about 70 weight percent to about 80 weight percent, from about 80 weight percent to about 90 weight percent, or from about 90 weight percent to about 100 weight percent. In one embodiment, a reaction product may include a composition having formula (I), unreacted polynorbornene, and unreacted composition having formula (XIV).

In one embodiment, a method is provided. The method includes contacting a composition having a formula (XIV) with an ionic group-substituted cycloolefin or an ionic group-substituted polynorbornene. In one embodiment, a composition having formula (XIV) may be contacted with a cycloolefin or a polynorbornene in a nonpolar solvent. In one embodiment, a composition having formula (XIV) may be contacted with a cycloolefin or a polynorbornene in a polar solvent. In one embodiment, a composition having formula (XIV) may be contacted with a cycloolefin or a polynorbornene in a mixture of a polar solvent and a nonpolar solvent. In one embodiment, a composition having formula (XIV) may be contacted with a cycloolefin or a polynorbornene in a solution. In one embodiment, a composition having formula (XIV) may be contacted with a cycloolefin or a polynorbornene in a suspension. In one embodiment, a composition having formula (XIV) may be contacted with a cycloolefin or a polynorbornene in an emulsion.

A polar solvent may include one or more of water, methanol, tetrahydrofuran, ethanol, isopropanol, ethylene glycol, 1,4-dioxane, morpholine, dimethyl sulfoxide, dimethyl formamide, acetone, acetonitrile, nitrile, nitromethane, pyridine, dimethyl pyridine, or N-methyl pyrrolidinone. A non-polar solvent may include one or more of hexane, toluene, benzene, ethyl ether, chloroform, dichloromethane, or ethyl acetate.

In one embodiment, a method may include contacting a composition having formula (XIV) with a nonpolar solvent to form a solution or a slurry. In one embodiment, a method may include contacting a cycloolefin or a polynorbornene with a polar solvent to form a solution or slurry. In one embodiment, a method may include contacting a solution of the composition having formula (XIV) with a solution of a cycloolefin or a polynorbornene. In one embodiment, amount of the polar solvent to the amount of the non polar solvent in the resulting mixture may be varied in a range of from about 1:4 to about 4:1.

In one embodiment, a method may include contacting the composition having a formula (XIV) with a cycloolefin or a polynorbornene at room temperature. In one embodiment, a method may include contacting the composition having a formula (XIV) with a cycloolefin or a polynorbornene at a temperature in a range of from about −80 degrees Celsius to about −75 degrees Celsius, from about −75 degrees Celsius to about −50 degrees Celsius, from about −50 degrees Celsius to about −25 degrees Celsius, from about −25 degrees Celsius to about 0 degrees Celsius, or from about 0 degrees Celsius to about 25 degrees Celsius.

In one embodiment, a method may include reacting the composition having a formula (XIV) with an ionic group-substituted cycloolefin or an ionic group-substituted polynorbornene to form a metal complex having formula (I). In one embodiment, a method may include initiating a ring opening metathesis reaction of the ionic group-substituted cycloolefin using the composition having formula (XIV) to form a metal complex having formula (I). In one embodiment, a method may include initiating an alkene displacement reaction of the ionic group-substituted polynorbornene using the composition having formula (XIV) to form a metal complex having formula (I).

In one embodiment, a method may include controlling the chain length or “n” by varying one or more of molar ratio of the composition having formula (XIV) and the ionic group-substituted cycloolefin or the ionic group-substituted polynorbornene, the reaction temperature, or the time duration of reaction. In one embodiment, a method may include controlling the chain length or “n” by varying a molar ratio of the composition having formula (XIV) and the cycloolefin or the polynorbornene. In one embodiment, a molar ratio of composition having formula (XII) and the cycloolefin or the polynorbornene may be varied to control “n” to be in a range of from about 10 to about 20.

In one embodiment, a method may include contacting a composition having formula (XIV) with an ionic group-substituted cycloolefin or an ionic group-substituted polynorbornene to form a composition having formula (I) that is soluble in a polar solvent. In one embodiment, a composition is provided that includes a metal complex having formula (I) and a polar solvent.

A polar solvent may be present in an amount sufficient that the composition is a slurry (a suspension) or a solution. In one embodiment, a polar solvent may be present in an amount sufficient that the composition is a solution. In one embodiment, a polar solvent may be present in an amount sufficient that the composition may not phase separate at room temperature. In one embodiment, a polar solvent may be present in an amount sufficient that the composition may be substantially transparent to visible light. The term “transparent” may refer to allowing at least 50 percent, at least 80 percent, or at least 90 percent, of incident light in the visible wavelength range having an angle of incidence of less than about 10 degrees to be transmitted.

Solubility of a metal complex in a polar solvent may be affected by one or more of the type of ionic groups, type of polar solvent, number of ionic groups in the metal complex, molecular weight of the metal complex, concentration of metal complex in the polar solvent, or temperature. In one embodiment, solubility of a metal complex n in the polar solvent may be adjusted by varying the chain length or “n”. In one embodiment, solubility of a metal complex in the polar solvent may be adjusted by varying the number of ionic groups present in the metal complex. In one embodiment, “n” may be in a range of from about 10 to about 20 and the metal complex may form a solution in the polar solvent.

In one embodiment, a metal complex may have a room temperature solubility in a polar solvent in a range of greater than about 1 gram per liter. In one embodiment, a metal complex may have a room temperature solubility in a polar solvent in a range of from about 1 gram per liter to about 5 grams per liter, from about 5 grams per liter to about 10 grams per liter, from about 10 grams per liter to about 20 grams per liter, from about 20 grams per liter to about 30 grams per liter, from about 30 grams per liter to about 40 grams per liter, from about 40 grams per liter to about 60 grams per liter, from about 60 grams per liter to about 80 grams per liter, or from about 80 grams per liter to about 100 grams per liter. In one embodiment, a metal complex may have a room temperature solubility in a polar solvent in a range of greater than about 100 grams per liter.

In one embodiment, a composition is provided that includes a metal complex, a polar solvent, and an olefin. An olefin or an alkene refers to an organic compound having at least one carbon-carbon double bond. In one embodiment, the olefin is soluble in the polar solvent, that is, the polar solvent is present in an amount sufficient such that the composition is a solution. In one embodiment, the polar solvent is present in an amount sufficient such that the metal complex and the olefin form a homogeneous solution. In one embodiment, the olefin is insoluble in the polar solvent, and the composition is a slurry.

In one embodiment, an emulsion is provided. An emulsion includes a first phase and a second phase. The first phase includes an olefin. The second phase includes a polar solvent and a metal complex. The polar solvent is present in an amount sufficient such that metal complex is dissolved in the polar solvent to form a continuous phase. In one embodiment, a first phase includes a non-polar solvent, and the olefin is soluble in non-polar solvent. In one embodiment, the first phase is dispersed in the second phase that is a continuous phase. In one embodiment, a composition may include a surfactant. A surfactant may stabilize the emulsion.

In one embodiment, a metal complex may initiate a metathesis reaction when contacted to an olefin. A metathesis reaction of an olefin refers to a chemical reaction involving redistribution of alkene bonds. In one embodiment, a metal complex may initiate one or more of a cross-metathesis reaction, a ring-closing metathesis reaction, a ring-opening metathesis reaction, or an acyclic diene metathesis reaction when contacted to an olefin.

In one embodiment, an olefin may include an acyclic olefin. In one embodiment, an olefin may include a cyclic olefin (or cycloolefin). A cycloolefin may be strained or unstrained. In one embodiment, an olefin may include two or more carbon-carbon double bonds, for example, dienes. In one embodiment, an olefin may include one or more functional groups either as substituents of the olefins or incorporated into the carbon chain of the olefin. Suitable functional groups may include one or more of alcohol, thiol, ketone, aldehyde, ester, disulfide, carbonate, imine, carboxyl, amine, amide, nitro acid, carboxylic acid, isocyanate, carbodiimide, ether, halogen, quaternary amine, carbohydrate, peptide, phosphate, sulfate or sulfonate.

In one embodiment, a composition may include two or more acyclic olefins, a polar solvent, and a metal complex. A metal complex may initiate a cross-metathesis reaction of the acyclic olefins. In one embodiment, a composition may include a cycloolefin, an acyclic olefin, a polar solvent, and a metal complex. A metal complex may initiate a ring-opening cross-metathesis reaction of a cycloolefin and an acyclic olefin. In one embodiment, a composition may include a cycloolefin, a polar solvent, and a metal complex. A metal complex may initiate a ring-opening metathesis reaction of the cycloolefin.

In one embodiment, a composition may include a cycloolefin polymer precursor, a polar solvent, and the metal complex. A metal complex may initiate a ring-opening metathesis polymerization reaction of the cycloolefin polymer precursor. A polymer precursor may include monomeric species, oligomeric species, mixtures of monomeric species, mixtures of oligomeric species, polymeric species, mixtures of polymeric species, partially-crosslinked species, mixtures of partially-crosslinked crosslinked species, or mixtures of two or more of the foregoing. In one embodiment, a metal complex may be particularly suitable for ring opening metathesis polymerization of a cycloolefin polymer precursor in a polar solvent, such as water. In one embodiment, a metal complex may be particularly suitable for ring opening metathesis polymerization of a hydrophilic cycloolefin polymer precursor in a polar solvent, such as water.

In one embodiment, a cycloolefin polymer precursor may include one or more of norbornene, dicyclopentadiene, di(methyl) dicyclopentadiene, dilhydrodicyclopentadiene, cyclopentadiene trimer, cyclopentadiene tetramer, tetracyclododecene, ethylidenenorborniene, methyltetracyclododecene, methylnorborinene, ethylnorbornene, dimethylnorbornene, norbornadiene, cyclopentene, cycloheptene, cyclooctene, 7-oxanorbornene, 7-oxabicyclo[2.2.1]hept-5ene derivatives, 7-oxanorbornadiene, cyclododecene, 2-norbornene (also named bicyclo[2.2.1]-2-heptene), 5-methyl-2-norbornene, 5,6-dimethyl-2-norbornene, 5-ethyl-2-norbornene, 5-butyl-2-norbornene, 5-hexyl-2-norbornene, 5-dodecyl-2-norbornene, 5-isobutyl-2-norbornene, 5-octadecyl-2-norbornene, 5-isopropyl-2-norbornene, 5-phenyl-2-norbornene, 5-p-toluyl-2-norbornene, 5-a-naphthyl-2-norbornene, 5-cyclohexyl-2-norbornene, 5,5-dimethyl-2-norbornene, dicyclopentadiene (or cyclopentadiene dimer), dihydrodicyclopentadiene (or cyclopentene cyclopentadiene codimer), methyl-cyclopentadiene dimer, ethyl-cyclopentadiene dimer, tetracyclododecene (also named 1,2,3,4,4a,5,8,8a-octahydro-1,4:5,8-dimethyanonaphthalene), 9-methyl-tetracyclo[6.2.1.1^{3,6}.0^{2,7}]-4-dodecene (also named 1,2,3,4,4a,5,8,8a-octahydro-2-metlhyl-4,4:5,8-dimethanonaphthalene), 9-ethyl-tetracyclo[6.2.1.1^{3,6}.0^{2,7}]-4-dodecene, 9-propyl-tetracyclo[6.2.1.1^{3,6}.0^{2,7}]-4-dodecene, 9-hexyl-tetracyclo[6.2.1.1^{3,6}.0^{2,7}]-4-dodecene, 9-decyl-tetracyclo[6.2.1.1^{3,6}.0^{2,7}]-4-dodecene, 9,10-dimethyl-tetracyclo[6.2.1.1^{3,6}.0^{2,7}]-4-dodecene, 9-ethlyl, 10-methyl-tetracyclo[6.2.1.1^{3,6}.0^{2,7}]-4-dodecene, 9-cyclohexyl-tetracyclo[6.2.1.1^{3,6}.0^{2,7}]-4-dodecene, 9-chloro-tetracyclo[6.2.1.1^{3,6}.0^{2,7}]-4-dodecene, 9-bromo-tetracyclo[6.2.1.1^{3,6}.0^{2,7}]-4-dodecene, cyclopentadiene-trimer, methyl-cyclopentadiene-trimer, or derivatives of the foregoing. In one embodiment, a cycloolefin polymer precursor may include one or more functional groups either as substituents of the cycloolefin polymer precursor or incorporated into the carbon chain of the cycloolefin polymer precursor.

In one embodiment, a cycloolefin polymer precursor may include at least one norbornene group having formula (XIX)

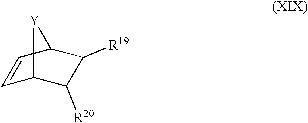

wherein R¹⁹ and R²⁰ are independently one or more of alcohol, thiol, ketone, aldehyde, ester, disulfide, carbonate, imine, carboxyl, amine, amide, nitro acid, carboxylic acid, isocyanate, carbodiimide, ether, halogen, quaternary amine, carbohydrate, peptide, phosphate, sulfate, sulfonate, or R¹⁹ and R²⁰ together form a cycloaliphatic radical, an aromatic radical, or a divalent bond linking two carbon atoms; and Y is C(R²¹)₂, C═C(R²¹)₂, Si(R²¹)₂, O, S, N—R²¹, P—R³¹, B—R²¹, or As—R²¹, wherein R²¹ is independently at each occurrence hydrogen, an aliphatic radical, a cycloaliphatic radical, or an aromatic radical. Aliphatic radical, cycloaliphatic radical, and an aromatic radical are as defined hereinabove. Peptide and carbohydrate are as defined hereinabove.

In one embodiment, at least one of R¹⁹ or R²⁰ may include a peptide group, a carbohydrate group, or derivatives of the foregoing. In one embodiment, at least one of R¹⁹ or R²⁰ may include a derivative of glucose. In one embodiment, at least one of R¹⁹ or R²⁰ may include a carboxylic acid derivative of glucose. In one embodiment, at least one of R¹⁹ or R²⁰ may include a gluconate group.

In one embodiment, a metathesis method is provided. In one embodiment, a metathesis method may include contacting a metal complex with an olefin. In one embodiment, a metal complex may be contacted with an olefin in a polar solvent. In one embodiment, a metal complex may be contacted with an olefin in a non-polar solvent. In one embodiment, a metal complex may be contacted with an olefin in a mixture of a polar solvent and a non-polar solvent. In one embodiment, a metal complex may be essentially contacted with an olefin in a polar solvent.

In one embodiment, a metathesis polymerization method is provided. In one embodiment, a metathesis polymerization method may include contacting a metal complex with a cycloolefin polymer precursor. In one embodiment, a metal complex may be contacted with a cycloolefin polymer precursor in a polar solvent. In one embodiment, a metal complex may be contacted with a cycloolefin polymer precursor in a non-polar solvent. In one embodiment, a metal complex may be contacted with a cycloolefin polymer precursor in a mixture of a polar solvent and a non-polar solvent. In one embodiment, a metal complex may be essentially contacted with a cycloolefin polymer precursor in a polar solvent.

In one embodiment, a reaction between a metal complex and an olefin may be effected in a polar solvent. In one embodiment, a reaction between a metal complex and an olefin may be effected in a solution. In one embodiment, a reaction between a metal complex and an olefin may be effected in a suspension. In one embodiment, a reaction between a metal complex and an olefin may be effected in an emulsion.

In one embodiment, a metal complex may initiate a ring opening metathesis polymerization reaction when contacted to a cycloolefin polymer precursor. Initiator efficiency or catalytic efficiency of the metal complex composition may be affected by one or more of the reaction conditions (for example, temperature, solvent, and the like), number of ligands, type of ligands, or type of alkylidene ligand substituents.

Initiator efficiency or catalytic efficiency of the metal complex may be measured as the turnover frequency (TOF) of the polymer precursor. Turnover frequency refers to the number of polymer precursors converted by the metal complex in one second. In one embodiment, a metal complex may have a polymer precursor turnover frequency in a range of greater than about 10 per second. In one embodiment, a metal complex may have a turnover frequency in a range of from about 10 per second to about 20 per second, from about 20 per second to about 50 per second, from about 50 per second to about 100 per second, from about 100 per second to about 250 per second, from about 250 per second to about 500 per second, from about 500 per second to about 1000 per second, from about 1000 per second to about 1200 per second, from about 1200 per second to about 1400 per second, from about 1400 per second to about 1600 per second, from about 1600 per second to about 1800 per second, or from about 1800 per second to about 2000 per second. In one embodiment, a metal complex may have a polymer precursor turnover frequency in a range of greater than about 2000 per second.

In one embodiment, a metal complex may be stable in a polar solvent. Stability of a metal complex, as used herein, refers to the ratio of turnover frequency of the polymer precursor measured initially, and measured again after a period of time, e.g., one week, two weeks, and the like. In one embodiment, a metal complex may be stable at room temperature in a polar solvent for a time period in a range that is greater than about 1 day. In one embodiment, a metal complex may be stable at room temperature in a polar solvent for a time period in a range of from about 1 day to about 2 days, from about 2 days to about 5 days, from about 5 days to about 10 days, from about 10 days to about 30 days, from about 30 days to about 60 days, from about 60 days to about 120 days, from about 120 days to about 240 days. In one embodiment, a metal complex may be stable at room temperature in a polar solvent for a time period in a range that is greater than about 240 days.

In one embodiment, a composition may include a reaction product of a metal complex and an olefin. In one embodiment, a composition may include a polymeric reaction product of a metal complex and a cycloolefin polymer precursor. The polymer formed may be a homopolymer or a copolymer. The polymer formed may be linear, branched, or crosslinked. In one embodiment, a polymeric reaction product may be soluble in the polar solvent. In one embodiment, a polymeric reaction product may be insoluble in the polar solvent.

In one embodiment, the conversion of the polymer precursor may be complete, that is, the reaction product may be free of any unreacted polymer precursor. In one embodiment, the conversion of the polymer precursor may be incomplete, that is, the reaction product may include unreacted polymer precursor. In one embodiment, the conversion of the polymer precursor may be in a range that is greater than about 50 weight percent. In one embodiment, the conversion of the polymer precursor may be in a range of from about 50 weight percent to about 60 weight percent, from about 60 weight percent to about 70 weight percent, from about 70 weight percent to about 80 weight percent, from about 80 weight percent to about 90 weight percent, or from about 90 weight percent to about 100 weight percent.

In one embodiment, a metal complex may be immobilized on a solid-support prior to contacting with the cycloolefin polymer precursor. A solid support may include a polymeric, a silica, or a metallic material. In one embodiment, a solid support may include a membrane, a microtiter plate, a bead, a filter, a test strip, a slide, a cover slip, a chip, a nanoparticle, or a test tube. A metal complex having formula (I) may be immobilized on the solid support by physical adsorption, by covalent bond formation, or by combinations thereof.

In one embodiment, a method may include immobilizing a metal complex on a surface of a solid support by physical adsorption, by covalent bond formation, or by combinations thereof. In one embodiment, a method may include contacting the immobilized metal complex with a cycloolefin polymer precursor. In one embodiment, a method may include contacting the immobilized metal complex with a cycloolefin polymer precursor in a polar solvent. In one embodiment, a method may include contacting the immobilized metal complex with a cycloolefin polymer precursor in a non-polar solvent. In one embodiment, a method may include contacting the immobilized metal complex with a cycloolefin polymer precursor in a mixture of a polar solvent and a non- polar solvent.

In one embodiment, an article is provided. The article may include a solid-support and a metal complex immobilized on a surface of the solid support. In one embodiment, an article may include a solid-support and a polymeric reaction product of a metal complex and a cycloolefin polymer precursor, immobilized on a surface of the solid support.

In one embodiment, the compositions and methods described hereinabove may be useful in one or more bioanalytical, pharmaceutical, or polymer synthesis applications.

In one embodiment, the compositions and methods described hereinabove may be used to prepare pharmaceutical intermediates by ring closing metathesis reactions or cross metathesis reactions. In one embodiment, the compositions and methods disclosed hereinabove may be used to prepare biopolymers such as glycopolymers or other biomolecule-functionalized polymers. In one embodiment, the compositions and methods disclosed hereinabove may be used to prepare core-shell polymer particles that may be applicable for drug delivery. In one embodiment, the compositions and methods disclosed hereinabove may be used to prepare monolithic chromatographic separation media.

In one embodiment, the compositions and methods disclosed hereinabove may be used to prepare membrane material. In one embodiment, the compositions and methods disclosed hereinabove may be used to prepare ion-exchange resin. In one embodiment, the compositions and methods disclosed hereinabove may be used to prepare molecular-imprinted separation material. In one embodiment, the compositions and methods disclosed hereinabove may be used to prepare bulk-engineering plastic.

### EXAMPLES

The following examples are intended only to illustrate methods and embodiments in accordance with the invention, and as such should not be construed as imposing limitations upon the clauses.

### Example 1

### Synthesis of norbornene-sodium sulfonate

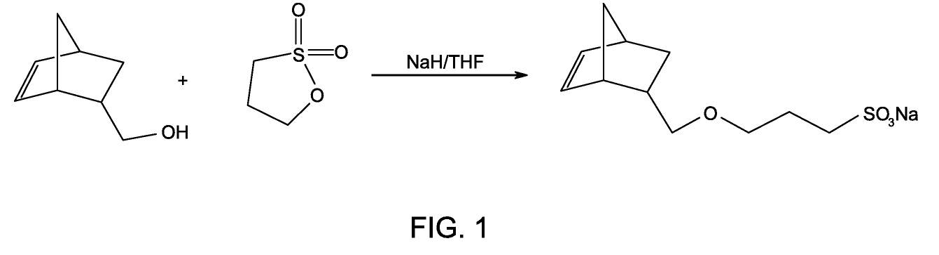

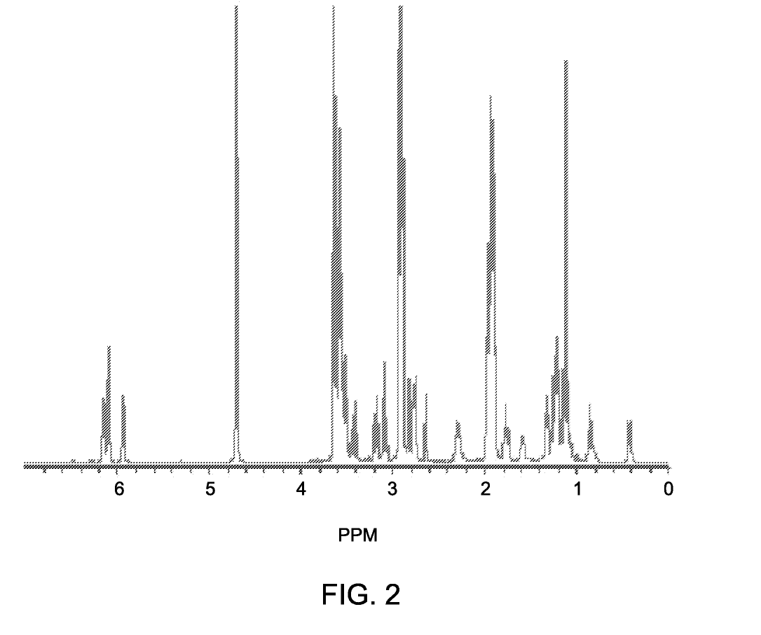

5-norbornene-2-methanol (CAS 95-12-5, 1 gram) and sodium hydride (NaH, 60% dispersion in mineral oil, 0.6 grams) are dissolved in 50 milliliters of anhydrous THF. The mixture is refluxed overnight at 80 degrees C. After refluxing, 1,3-propane sultone (CAS 1120-71-4, 0.98 grams) in 20 milliliters of THF is added to the mixture in 10 minutes. After 5 hours, the solvent is removed by rotary evaporation and the reaction product is purified by column chromatography using silica and ethyl acetate/methanol (4:1) as eluent. The purified reaction product is a white solid and is soluble in water or methanol. The product yield is about 60 weight percent. FIG. 1 shows the reaction scheme and FIG. 2 shows the proton NMR spectrum of norbornene-sodium sulfonate (Sample 1).

### Example 2

### Synthesis of norbornene-gluconate

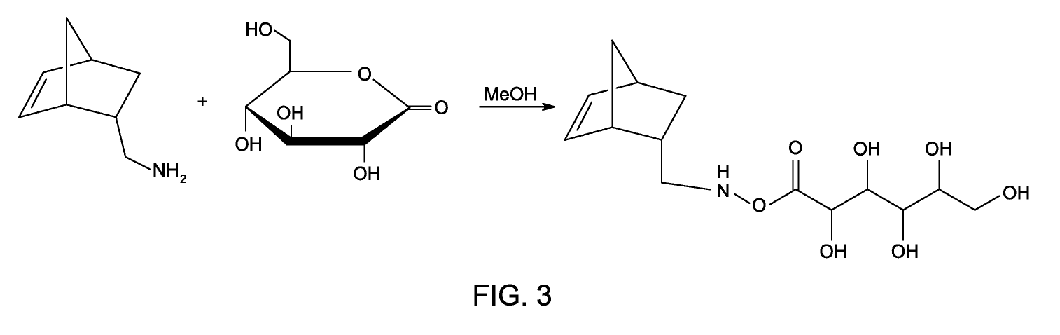

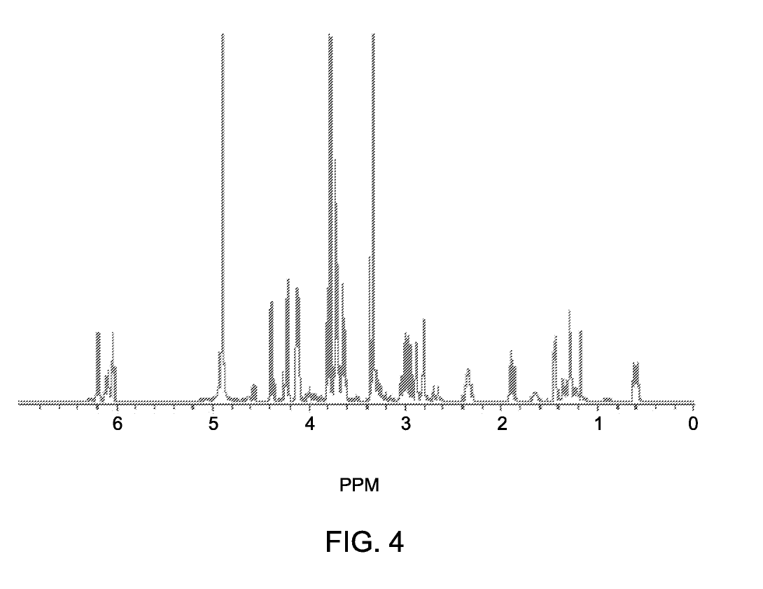

Methanol (2 milliliters) is added to a 50 milliliters flask containing 1 gram of glucolactone (purchased from Aldrich, and used directly). Norbornenyl-methylamine (prepared by the reduction of norbornene carbonitrile with LiAlH4, 0.74 grams) is added to the flask and solid glucolactone disappears gradually within 20 minutes. The reaction is carried on overnight at room temperature. After the reaction is complete, methanol is removed by rotary evaporation. The reaction product is recrystallized from iPrOH/petroleum ether at −4 degrees Celsius. The product yield is about 70 weight percent. FIG. 3 shows the reaction scheme and FIG. 4 shows the proton NMR spectrum of norbornene gluconate (Sample 2).

### Example 3

### Synthesis of water-soluble ruthenium complex

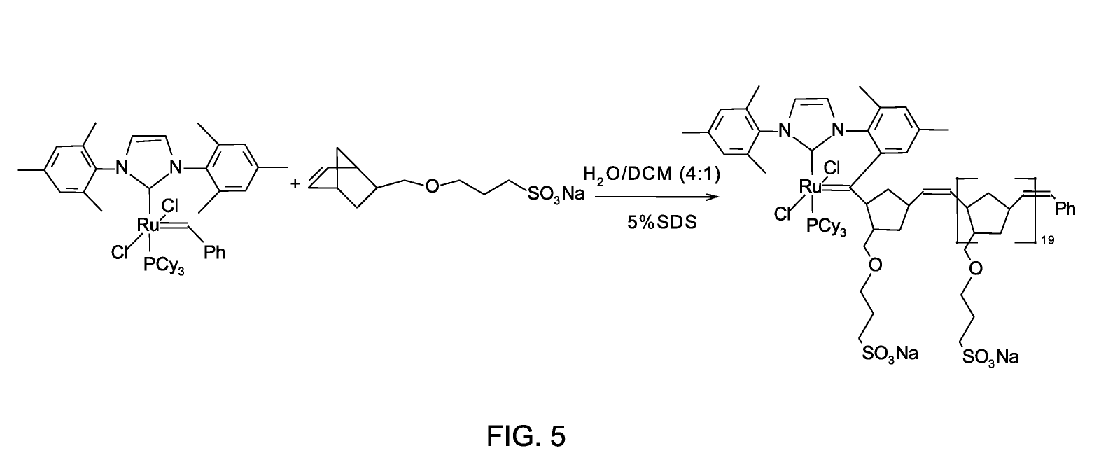

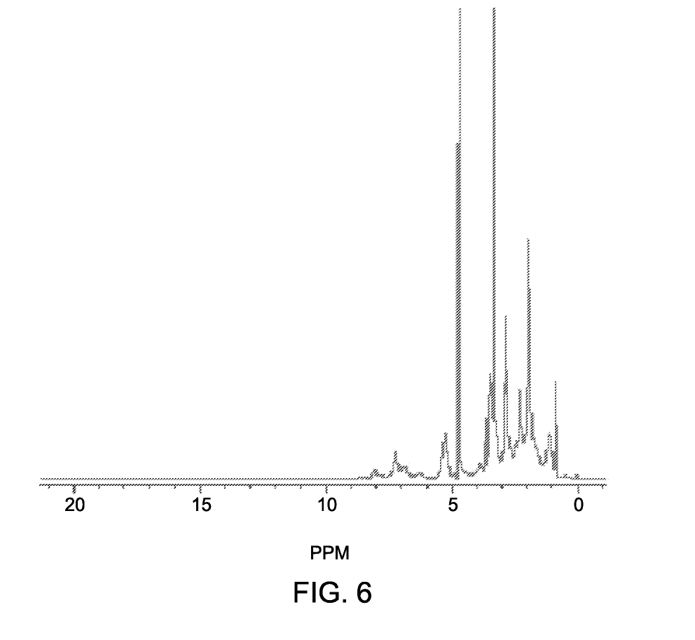

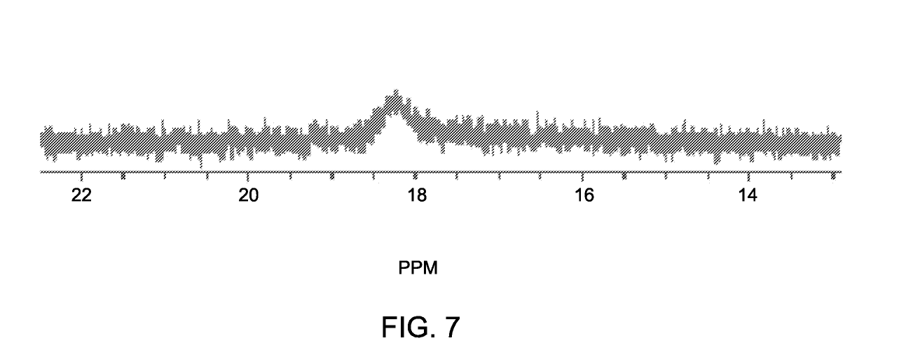

A ruthenium metal complex 1,3-bis(2,4,6-trimethylphenyl)-2-imidazolidinylidene)dichloro(phenylmethylene)(tricyclohexylphosphine)ruthenium (CAS No. 246047-72-3, 5 milligrams) is charged in a HPLC flask and the flask is degassed and purged with argon three times. Methylene chloride (CH₂Cl₂, 50 microliters) is injected into the flask using a syringe. The metal complex solution is cooled at −78 degrees Celsius in a dry ice and acetone bath. Norbornene sulfonate (10 milligrams, Sample 1 from Example 1) is charged into a second flask and degassed and the flask is backfilled with argon three times. Degassed methanol (500 microliters) is added to norbornene sulfonate using a syringe. The resulting mixture is also cooled at −78 degrees Celsius. After cooling for 5 minutes, the norbornene sulfonate mixture is transferred into the metal complex solution using a syringe. The resulting mixture is allowed to mix at −78 degrees Celsius and then the mixture is allowed to sit at room temperature for about 10 minutes. A yellow precipitate is observed after about 2 minutes. After the reaction is complete, the solvent is removed and a bright yellow water-soluble ruthenium complex is obtained. FIG. 5 shows the reaction scheme and FIG. 6 shows the proton NMR spectrum of the water-soluble ruthenium complex. FIG. 7 shows the broad proton NMR signal around 18 ppm for the alkylidene hydrogen of the water-soluble ruthenium complex (Sample 3).

Ruthenium metal complexes 1,3-bis-(2,4,6-trimethylphenyl)-2-imidazolidinylidene)dichloro(o-isopropoxyphenylmethylene)ruthenium (CAS No. 301224-40-8) and 1,3-bis(2,4,6-trimethylphenyl)-2-imidazolidinylidene)dichloro(phenylmethylene)(di-3-bromopyridine)ruthenium (synthesized in-house) are used to synthesize water-soluble ruthenium complexes (Samples 4 and 5) using the procedure described hereinabove.

### Example 4

### ROMP catalyzed by the water-soluble ruthenium complex

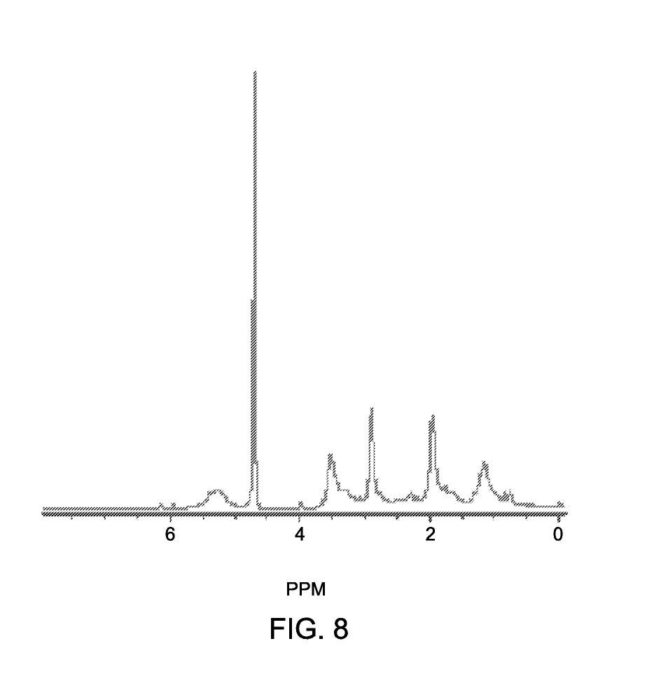

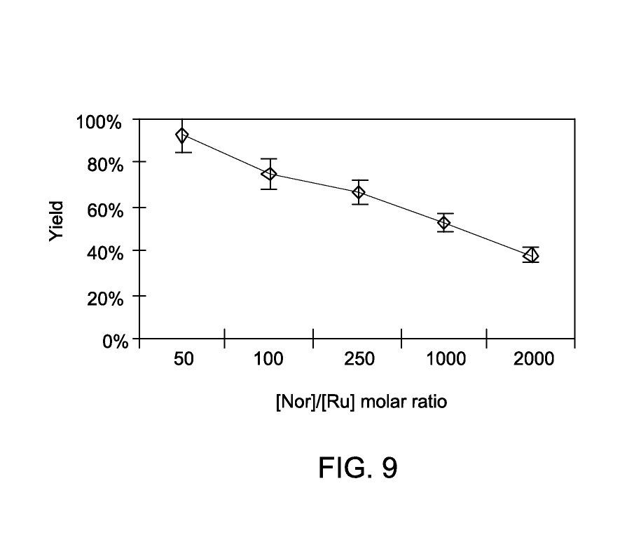

The ruthenium complex (Sample 3, corresponding to monomer/catalyst 50 molar ratio) is dissolved in water (0.5 milliliters) and a water-soluble monomer norbornene-gluconate (Sample 2 from Example 2, 20 milligrams) is added to the ruthenium complex solution. The solution solidifies almost instantaneously and a solid reaction product is obtained after about 20 minutes. The reaction product is precipitated from excess ethanol. FIG. 8 shows the proton NMR spectra of the polymer formed by ROMP of norbornene gluconate initiated by the water-soluble ruthenium complex synthesized in Example 3. The monomer to catalyst molar ratio is varied in a range of 50 to 2000 and the reaction product yield is calculated. The effect of monomer/catalyst molar ratio on the polymer yield is depicted in FIG. 9. The highest turn over frequency observed for the water-soluble ruthenium catalysts is around 1000 s⁻¹.

### Example 5

### Preparation of surface-supported water soluble ruthenium complex on quartz surface

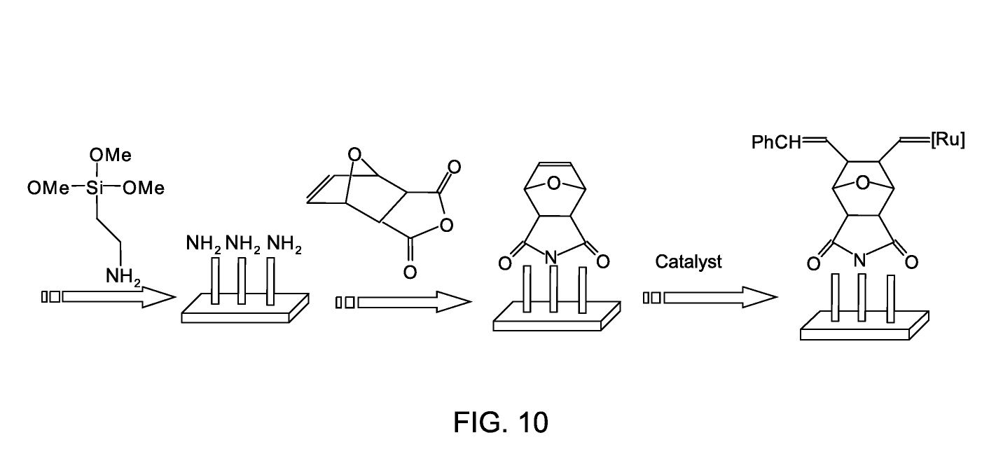

Quartz slides are modified with amino propyl trimethoxy silane (at 1% (v/v) and 10% (v/v) concentration) as shown in FIG. 10. Amine-functionalized quartz slides are reacted with 7-oxanorbornene-2,3-dicarboxylic anhydride to immobilize norbornene on the surface. Norbornene-functionalized quartz slides are then reacted with a water-soluble ruthenium complex (Sample 3 from Example 3) to form surface-supported water soluble ruthenium complex on quartz surface (Samples 6a and 6b). Sample 6a corresponds to 1% amine concentration on quartz surface and Sample 6b corresponds to 10% amine concentration on quartz surface.

Water-soluble ruthenium complex (Sample 4 from Example 3) is used to prepare surface-supported water soluble ruthenium complex on quartz surface (Samples 7a and 7b) using the procedure described hereinabove. Sample 7a corresponds to 1% amine concentration on quartz surface and Sample 7b corresponds to 10% amine concentration on quartz surface.

Water-soluble ruthenium complex (Sample 5 from Example 3) is used to prepare to form surface-supported water soluble ruthenium complex on quartz surface (Samples 8a and 8b) using the procedure described hereinabove. Sample 8a corresponds to 1% amine concentration on quartz surface and Sample 8b corresponds to 10% amine concentration on quartz surface.

### Example 6

### Surface initiated polymerization of Cy5-labeled norbornene by water-soluble ruthenium complexes

The surface-immobilized water-soluble ruthenium complexes prepared in Example 5 are used to initiate a polymerization reaction of a monomer Cy5-norbornene in water. Quartz slide-surface coated with TMS is used as a control.

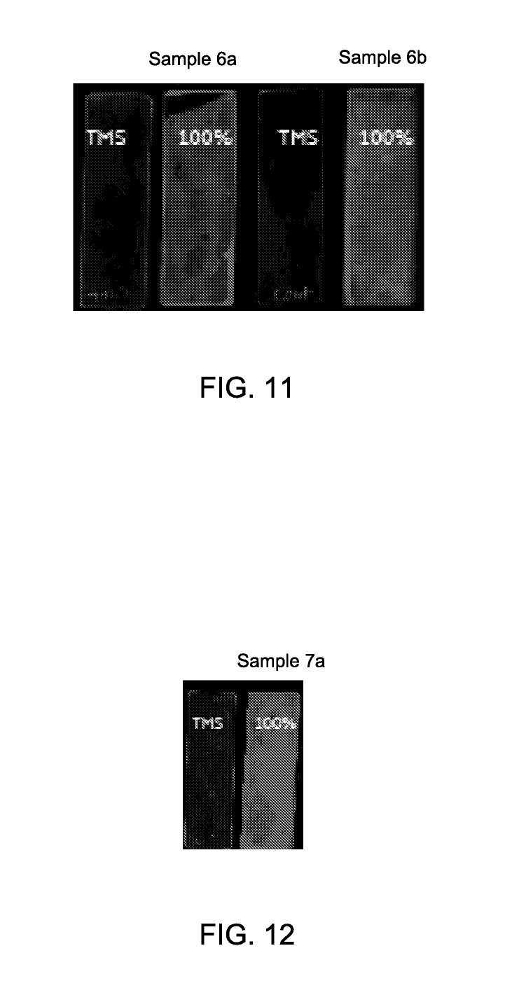

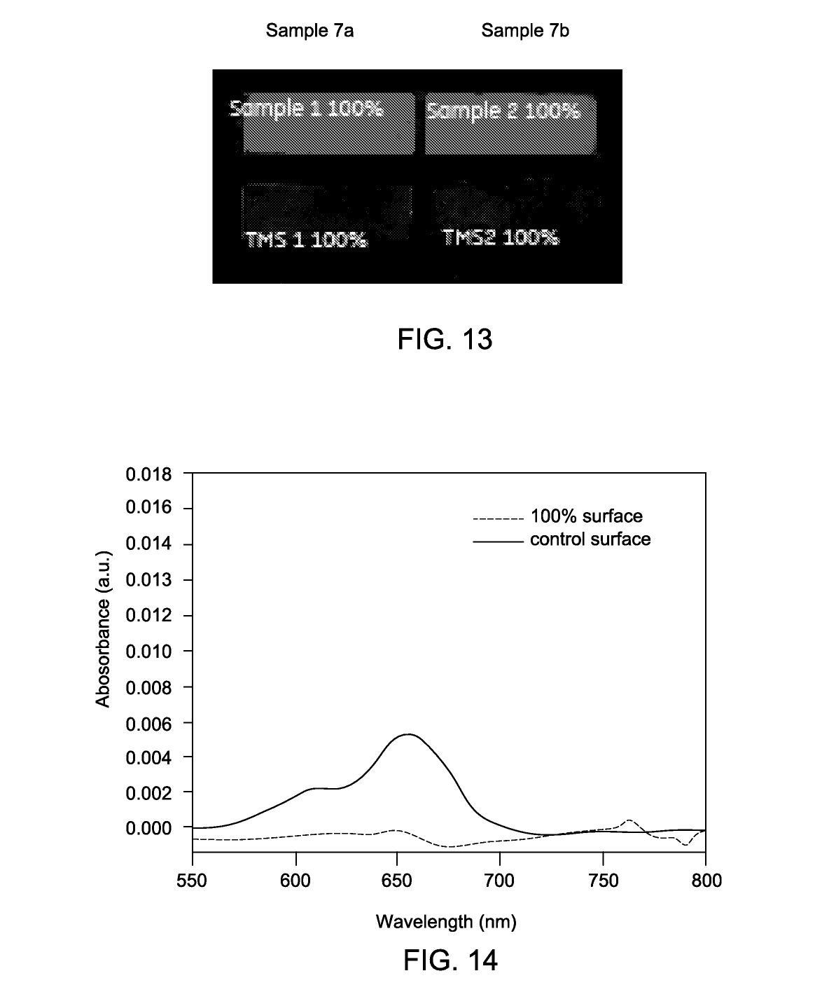

FIG. 11 shows the fluorescence images of Samples 6a and 6b versus the controls. FIG. 12 shows the fluorescence images of Sample 7a versus the control. FIG. 13 shows the fluorescence images of Samples 8a and 8b versus the controls. As shown in FIGS. 11-13, the catalytic activity of water-soluble ruthenium complexes follows the following trend: Sample 3<Sample 4<Sample 5, which is in good agreement with their precursors. FIG. 14 shows the UV-Vis absorption spectra of Sample 6a versus control depicting the effectiveness of water-soluble ruthenium complex in ROMP. Water-soluble ruthenium complexes also show better catalytic activity when compared to their water-insoluble ruthenium precursors.

Reference is made to substances, components, or ingredients in existence at the time just before first contacted, formed in situ, blended, or mixed with one or more other substances, components, or ingredients in accordance with the present disclosure. A substance, component or ingredient identified as a reaction product, resulting mixture, or the like may gain an identity, property, or character through a chemical reaction or transformation during the course of contacting, in situ formation, blending, or mixing operation if conducted in accordance with this disclosure with the application of common sense and the ordinary skill of one in the relevant art (e.g., chemist). The transformation of chemical reactants or starting materials to chemical products or final materials is a continually evolving process, independent of the speed at which it occurs. Accordingly, as such a transformative process is in progress there may be a mix of starting and final materials, as well as intermediate species that may be, depending on their kinetic lifetime, easy or difficult to detect with current analytical techniques known to those of ordinary skill in the art.

Reactants and components referred to by chemical name or formula in the specification or claims hereof, whether referred to in the singular or plural, may be identified as they exist prior to coming into contact with another substance referred to by chemical name or chemical type (e.g., another reactant or a solvent). Preliminary and/or transitional chemical changes, transformations, or reactions, if any, that take place in the resulting mixture, solution, or reaction medium may be identified as intermediate species, master batches, and the like, and may have utility distinct from the utility of the reaction product or final material. Other subsequent changes, transformations, or reactions may result from bringing the specified reactants and/or components together under the conditions called for pursuant to this disclosure. In these other subsequent changes, transformations, or reactions the reactants, ingredients, or the components to be brought together may identify or indicate the reaction product or final material.

The foregoing examples are illustrative of some features of the invention. The appended claims are intended to claim the invention as broadly as has been conceived and the examples herein presented are illustrative of selected embodiments from a manifold of all possible embodiments. Accordingly, it is Applicants' intention that the appended claims not limit to the illustrated features of the invention by the choice of examples utilized. As used in the claims, the word “comprises” and its grammatical variants logically also subtend and include phrases of varying and differing extent such as for example, but not limited thereto, “consisting essentially of” and “consisting of.” Where necessary, ranges have been supplied, and those ranges are inclusive of all sub-ranges there between. It is to be expected that variations in these ranges will suggest themselves to a practitioner having ordinary skill in the art and, where not already dedicated to the public, the appended claims should cover those variations. Advances in science and technology may make equivalents and substitutions possible that are not now contemplated by reason of the imprecision of language; these variations should be covered by the appended claims.

1. A composition, comprising
a Group (VIII) transition metal;
an anionic ligand bonded to the metal;
a neutral electron donor ligand bonded to the metal;
an alkylidene group bonded to the metal, and the alkylidene group comprises a cycloaliphatic radical substituted with an ionic group.

2. The composition as defined in claim 1, the composition having a formula (I): 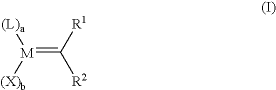
wherein “a” and “b” are independently integers from 1 to 3, with the proviso that “a+b” is less than or equal to 5;
M is ruthenium or osmium;
X is independently at each occurrence an anionic ligand;
L is independently at each occurrence a neutral electron donor ligand;
R¹ is hydrogen, an aliphatic radical, a cycloaliphatic radical, or an aromatic radical; and
R² comprises one or more cycloaliphatic radical, and the cycloaliphatic radical is substituted with an ionic group, wherein the ionic group comprises one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium.

3. The composition as defined in claim 2, wherein R² comprises structural units having a formula (II) or (III) 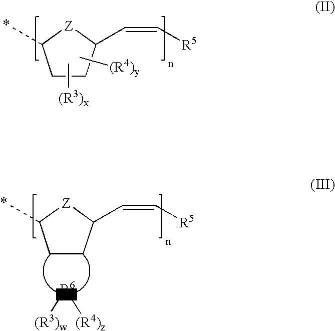
wherein “w” is 0, 1, 2, or 3, “x” is 0 or 1, “y” is 1 or 2, “z” is 1, 2, 3, or 4, “n” is an integer of from 1 to 100;
R³ is independently at each occurrence hydrogen, a halogen atom, an aliphatic radical, a cycloaliphatic radical, an aromatic radical, an alkoxy group, a hydroxy group, an ether group, an aldehyde group, a ketone group, a silanyl group, a phosphanyl group, an amine group, a nitro group, or a divalent bond linking two carbon atoms;
R⁴ is independently at each occurrence an ionic group, and the ionic group comprises one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium;
R⁵ is hydrogen, an aliphatic radical, a cycloaliphatic radical, or an aromatic radical;
R⁶ is an aliphatic or an aromatic cyclic ring; and
Z is C(R⁷)₂, C═C(R⁷)₂, Si(R⁷)2, O, S, N—R⁷, P—R⁷, B—R⁷, or As—R⁷ wherein R⁷ is independently at each occurrence hydrogen, an aliphatic radical, a cycloaliphatic radical, or an aromatic radical.

4. The composition as defined in claim 3, wherein “n” is an integer in a range of from about 10 to about 20.

5. The composition as defined in claim 2, wherein R² comprises structural units having a formula (IX) or (X) 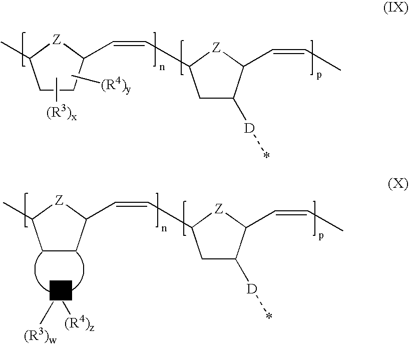
wherein “w” is 0, 1, 2, or 3, “x” is 0 or 1, “y” is 1 or 2, “z” is 1, 2, 3, or 4, “n” is an integer of from 1 to 100, “p” is an integer of from 1 to 100;
R³ is independently at each occurrence hydrogen, a halogen atom, an aliphatic radical, a cycloaliphatic radical, an aromatic radical, an alkoxy group, a hydroxy group, an ether group, an aldehyde group, a ketone group, a silanyl group, a phosphanyl group, an amine group, a nitro group, or a divalent bond linking two carbon atoms;
R⁴ is independently at each occurrence an ionic group, and the ionic group comprises one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium;
R⁶ is an aliphatic or an aromatic cyclic ring;
Z is C(R⁷)₂, C═C(R⁷)₂, Si(R⁷)2, O, S, N—R⁷, P—R⁷, B—R⁷, or As—R⁷ wherein R⁷ is independently at each occurrence hydrogen, an aliphatic radical, a cycloaliphatic radical, or an aromatic radical; and
D is a divalent aromatic radical.

6. The composition as defined in claim 5, wherein “n+p” is an integer in a range of from about 10 to about 20.

7. The composition as defined in claim 2, wherein X is independently at each occurrence a halide, a carboxylate, a sulfonate, a sulfonyl, a sulfinyl, a diketonate, an alkoxide, an aryloxide, a cyclopentadienyl, a cyanide, a cyanate, a thiocyanate, an isocyanate, or an isothiocyanate.

8. The composition as defined in claim 2, wherein at least one L is phosphine, phosphite, phosphinite, phosphonite, arsine, stibine, ether, amine, amide, imine, sulfoxide, carboxyl, nitrosyl, or thioethene.

9. The composition as defined in claim 2, wherein at least one L is a N-heterocyclic carbene ligand or a heteroarene ligand.

10. The composition as defined in claim 2, wherein at least one L is furan, thiophene, pyrrole, pyridine, bipyridine, picolylimine, gamma-pyran, gamma-thiopyran, phenanthroline, pyrimidine, bipyrimidine, pyrazine, indole, coumarone, thionaphthene, carbazole, dibenzofuran, dibenzothiophene, pyrazole, imidazole, benzimidazole, oxazole, thiazole, dithiazole, isoxazole, isothiazole, quinoline, bisquinoline, isoquinoline, bisisoquinoline, acridine, chromene, phenazine, phenoxazine, phenothiazine, triazine, thianthrene, purine, bisimidazole, or bisoxazole.

11. The composition as defined in claim 1, wherein the composition initiates a metathesis reaction when contacted to an olefin.

12. The composition as defined in claim 1, wherein the composition initiates a ring opening metathesis polymerization reaction when contacted to a cycloolefin polymer precursor.

13. The composition as defined in claim 1, wherein the composition has a room temperature solubility in a polar solvent in a range of from about 1 grams/liter to about 200 grams/liter.

14. The composition as defined in claim 1, wherein the composition has a polymer precursor turnover frequency in a range of from about 10 per second to about 2000 per second.

15. The composition as defined in claim 1, wherein the composition is stable in a polar solvent for a time period in a range that is greater than about 1 day.

16. A composition, comprising:
the composition as defined in claim 1; and
a polar solvent present in an amount sufficient that the composition is a slurry or a solution.

17. The composition as defined in claim 16, wherein the polar solvent comprises one or more of water, methanol, tetrahydrofuran, ethanol, isopropanol, ethylene glycol, 1,4-dioxane, morpholine, dimethyl sulfoxide, dimethyl formamide, acetone, acetonitrile, nitrile, nitromethane, pyridine, dimethyl pyridine, or N-methyl pyrrolidinone.

18. The composition as defined in claim 16, comprising an olefin soluble in the polar solvent.

19. A composition, comprising a reaction product of the composition as defined in claim 18.

20. A composition, comprising a polymeric reaction product of a cycloolefin polymer precursor with the composition as defined in claim 16.

21. An emulsion, comprising:
a first phase comprising an olefin; and
a second phase comprising a polar solvent and the composition as defined in claim 1.

22. The emulsion as defined in claim 21, wherein the first phase comprises a non-polar solvent.

23. The emulsion as defined in claim 21, comprising a surfactant.

24. The emulsion as defined in claim 21, wherein the first phase is dispersed in the second phase, and the second phase is a continuous phase.

25. A composition, comprising:
a reaction product of a composition having formula (XIV): 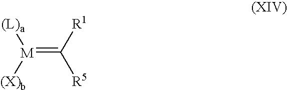
wherein “a” and “b” are independently integers from 1 to 3, with the proviso that “a+b” is less than or equal to 5,
M is ruthenium or osmium,
X is independently at each occurrence an anionic ligand,
L is independently at each occurrence a neutral electron donor ligand,
R¹ is hydrogen, an aliphatic radical, a cycloaliphatic radical, or an aromatic radical, and
R⁵ is hydrogen, an aliphatic radical, a cycloaliphatic radical, or an aromatic radical; and
a cycloolefin comprising an ionic group, or
a polynorbornene comprising an ionic group and a pendent alkene group, wherein the ionic group comprises one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium.

26. An article, comprising:
a substrate comprising a surface; and
the composition as defined in claim 1 adhered to the surface.

27. A method, comprising:
contacting a composition having formula (XIV) 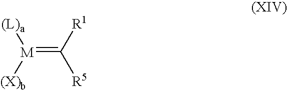
wherein “a” and “b” are independently integers from 1 to 3, with the proviso that “a+b” is less than or equal to 5,
M is ruthenium or osmium,
X is independently at each occurrence an anionic ligand,
L is independently at each occurrence a neutral electron donor ligand,
R¹ is hydrogen, an aliphatic radical, a cycloaliphatic radical, or an aromatic radical, and
R⁵ is hydrogen, an aliphatic radical, a cycloaliphatic radical, or an aromatic radical;
with a cycloolefin comprising an ionic group, or
a polynorbornene comprising an ionic group and a pendent alkene group, wherein the ionic group comprises one or more of phosphite, phosphate, sulfite, sulfate, sulfonate, nitrite, nitrate, azide, carboxylate, ammonium, quaternary ammonium, phosphonium, quaternary phosphonium, imidazolium, pyridinium, or sulfonium.

28. The method as defined in claim 26, comprising forming a catalyst soluble in a polar solvent.

29. The method as defined in claim 27, comprising contacting the catalyst with an olefin in a polar solvent.

30. The method as defined in claim 27, comprising contacting the catalyst with an olefin in a non-polar solvent.

31. The method as defined in claim 27, comprising contacting the catalyst with a cycloolefin polymer precursor and initiating a ring opening metathesis polymerization the cycloolefin polymer precursor.
